(19) United States
(12) Patent Application Publication
(10) Pub. No.: US 2005/0002873 A1
(43) Pub. Date: Jan. 6, 2005

(54) Therapeutic microfoam

(75) Inventors: Anthony David Harman, Henley-On-Thames (GB); David Dakin Iorwerth Wright, High Wycombe (GB)

Correspondence Address: NIXON & VANDERHYE, PC, 1100 N GLEBE ROAD, 8TH FLOOR, ARLINGTON, VA, 22201-4714, US

(73) Assignee: BTG International Limited, London (GB)

(21) Appl. No.: 10/890,267

(22) Filed: Jul. 14, 2004

Related U.S. Application Data
(63) Continuation-in-part of application No. PCT/GB04/00026, filed on Jan. 7, 2004.

(30) Foreign Application Priority Data
Jan. 10, 2003 (GB) 0300586.5

(51) Int. Cl. A61K 9/00

(52) U.S. Cl. 424/47

(57) ABSTRACT

A sclerosing foam comprising a physiologically acceptable gas that is readily dispersible in blood together with an aqueous sclerosant liquid is a microfoam further including one or more detectable gases selected from helium, neon, xenon, argon, sulfur hexafluoride and nitrous oxide, where the total volume of detectable gases comprises from 0.01% to 40% of the total volume of gas.

The present invention relates to a therapeutic microfoam comprising a sclerosing material, particularly a sclerosing liquid, which is suitable for use in the treatment of various medical conditions involving blood vessels, particularly varicose veins and other disorders involving venous malformation. The invention relates also to the method and apparatus for the generation of such a microfoam.

Sclerosis of varicose veins is based on the injection into the veins of liquid sclerosant substances which, by inter alia causing a localized inflammatory reaction, favor the elimination of these abnormal veins. Until recently, sclerotherapy was a technique selected in cases of small and medium caliber varicose veins, those with diameters equal to or greater than 7 mm being treated by surgery.

An injectable microfoam suitable for therapeutic use, on larger veins in particular, has now been developed and is described in EP-A-0656203 and U.S. Pat. No. 5,676,962 (incorporated herein by reference). These patents describe a low-density microfoam produced with a sclerosing substance which, when injected into a vein, displaces blood and ensures that the sclerosing agent contacts the endothelium of the vessel in a known concentration and for a controllable time, achieving sclerosis of the entire segment occupied.

The preparation of such a microfoam may be carried out with a solution of any sclerosing substance, particularly polidocanol. The method of preparation is to use a small brush attached to a high-speed motor to whip a dilute aqueous solution of the preferred sclerosant to a firm mousse-like consistency in a period of 1-2 minutes under a gas atmosphere containing physiologically acceptable gas mixes. However, this known method requires extemporaneous production of microfoam by the physician, pharmacist or an assistant immediately prior to administration to the patient. Such procedure allows for variation of microfoam sclerosing agent depending upon the person preparing it; microfoam density, gas makeup, bubble size and foam stability all needing attention with respect to the condition being treated.

A solution to this problem is offered in |WO 00/72821-A1| (BTG International Limited), incorporated herein by reference, which provides a method and a number of different devices that are capable of producing a uniform injectable microfoam. This microfoam is made with a relatively low concentration of a foamable sclerosing agent and a significant amount of a blood dispersible gas in sterile fashion without volatile liquid propellants or the need for the operator to directly be concerned in the control of its parameters. This application also addresses the perception that large volumes of nitrogen should not unnecessarily be introduced into patients. This is particularly an issue where large vessels are being filled with foam, if air is used as the gas for producing the foam. A preferred form of gas described in WO 00/72821-A1 comprises 50% vol/vol or more oxygen, the remainder being carbon dioxide, or carbon dioxide, nitrogen and trace gases in the proportion found in atmospheric air. Preferably the sclerosing agent is a solution of polidocanol or sodium tetradecyl sulfate in an aqueous carrier, e.g. water, particularly in a saline.

Various issues with long-term storage are not addressed in WO 00/72821-A1. One of these is a potential problem with storing the sclerosing fluid, for example, aqueous polidocanol, in the presence of oxygen. |WO 02/41872-A1| (BTG International Limited), incorporated herein by reference, offers a solution to this potential problem by storing the sclerosant liquid and the oxygen-rich physiologically acceptable blood dispersible gas in separate containers until immediately prior to use, when the blood-dispersible gas is introduced into the container holding the sclerosant liquid. The mixture of blood-dispersible gas and sclerosant liquid is then released, the components of the mixture interacting upon release of the mixture to form a sclerosing foam.

The present inventors have identified another issue with long-term storage of physiologically acceptable blood dispersible gases under pressure in a sealed canister, namely the need to ensure that potential leaks are minimized. They have determined that the introduction of a detectable gas, selected from helium, neon, xenon, argon, sulfur hexafluoride and nitrous oxide, into a physiologically acceptable blood dispersible gas gives a similarly physiologically acceptable mixture that is capable of being detected at very small quantities by a suitable sensor.

Accordingly the first aspect of the present invention provides a sclerosing foam comprising a physiologically acceptable gas that is readily dispersible in blood together with an aqueous sclerosant liquid, characterized in that the foam is a microfoam further including one or more detectable gases selected from helium, neon, xenon, argon, sulfur hexafluoride and nitrous oxide, where the total volume of detectable gases comprises from 0.01% to 40% of the total volume of gas.

Neon and argon are well known to be inert gases, sulfur hexafluoride (SF₆) is already used in echo contrast agents, and xenon can be used as a anesthetic agent. Nitrous oxide is also physiologically compatible. Although helium has very low solubility in water or blood, the helium gas molecules are small enough to readily diffuse across pulmonary gas exchange membranes and be exhaled. The safety of helium in respirable gas mixtures is well established and widely exploited (namely Heliox mixtures for deep sea divers containing up to 70% helium).

The advantage of helium in respirable gas mixtures results from its extremely low solubility in water or blood, even under high ambient pressures. Helium can also be shown to diffuse very rapidly across pulmonary gas exchange membranes, and therefore presents no danger of pulmonary gas embolism. Helium can also be used as an efficient marker of gas bubble arrival in the pulmonary circulation, following breakdown of a microfoam that has helium as a constituent gas.

Thus preferably the detectable gas comprises helium.

A commercially available leak detector (or “sniffer”) is the Veeco™ MS-40 portable automatic leak detector, provided by the Vacuum Instrument Corporation, Ronkonkoma, N.Y. This is said to detect a helium leakage level expressed in units of std cc/sec down to 4×10⁻¹¹, i.e. 4×10⁻¹¹ cm³s⁻¹ at standard temperature conditions.

In a typical device of the type disclosed in WO 00/72821-A1, a pressure loss of 0.15 bar in 3 years shelf life may be tolerated from a pressurized single-canister microfoam generator of 300 ml capacity, initially at 3.5 bar absolute, and containing 18 ml of a sclerosing liquid. Therefore the volume of gas lost from the canister in 3 years is given by V, where:

$$V = \frac{0.15}{1.00} \times ( 300 - 18 ) = 42.3 {cm}^{3}$$

This loss of 42.3 cm³ gas in 3 years corresponds to an average leak rate of:

$$\frac{42.3}{60 \times 60 \times 24 \times 365 \times 3} = 1.34 \times {10}^{- 6} {cm}^{3} {s}^{- 1}$$

Thus, if 3% helium were to be incorporated in the gas mixture, a leakage level of 3% of this figure, namely 4×10⁻⁸ cm³s⁻¹, would have to be detected. This is well within the ability of commercially available leak detectors such as the Veeco™ MS-40 portable automatic leak detector.

The limits of the present invention are a sclerosing foams including detectable gases in an amount from 0.01% to 40% of the total volume of gas. Similar calculations to the above show that the leakage level that has to be detected is 1×10⁻¹⁰ cm³s⁻¹ to 5×10⁻⁶ cm³s⁻¹, again within the ability of commercially available leak detectors.

Another commercially available leak detector is the Gas Check™ series provided by LDS Vacuum Products, Inc., Altamonte Springs, Fla. The minimum detection level is generally inversely proportional to the molecular weight, and with a typical device is:

| | |
|---|---|
| Helium | 2 × 10⁻⁵ cm³ s⁻¹ |
| SF₆ | 5 × 10⁻⁵ cm³ s⁻¹ |
| Neon | 9 × 10⁻⁵ cm³ s⁻¹ |
| Xenon | 1 × 10⁻⁵ cm³ s⁻¹ |
| Nitrous oxide | 2 × 10⁻⁴ cm³ s⁻¹ |
| Argon | 2 × 10⁻⁴ cm³ s⁻¹ |

Thus suitable sclerosing foams can be devised using one or more of the gases, depending on what rate of pressure loss is to be detected.

Preferably the microfoam includes detectable gases in an amount from 0.1% to 40% of the total volume of gas. More preferably the microfoam includes detectable gases in an amount from 0.5% to 20% of the total volume of gas. More preferably the microfoam includes detectable gases in an amount from 1% to 10% of the total volume of gas. More preferably the microfoam includes detectable gases in an amount from 1% to 5% of the total volume of gas.

The gas mixture may be regarded as made up of three components:
the physiologically acceptable gas or gases;
a detectable gas or gases; and optionally
a further inert gas or gases.

Suitable further inert gases include nitrogen. Preferably the gas mixture includes less than 10% vol/vol nitrogen. One or more gaseous perfluorocarbons may be included. Perfluorocarbons are well known for use in echo contrast. Gaseous perfluorocarbons include CF₄, C₂F₆ and C₃F₈.

Preferably the gas mixture comprises at least 50% of the physiologically acceptable gases oxygen and/or carbon dioxide, more preferably 75% or more oxygen and/or carbon dioxide and most preferably at least 99% oxygen or carbon dioxide. Preferably the oxygen or carbon dioxide is medical grade.

Further versatility may be achieved by using radiation-emitting isotopes of one or more of the components of the gas mixture. For example, molecular oxygen rich in ¹⁶O atoms could be used to detect leaks

In a second aspect of the present invention there is provided a method for producing a microfoam suitable for use in scleropathy of blood vessels, comprising introducing a physiologically acceptable blood-dispersible gas into a container holding an aqueous sclerosant liquid and releasing the mixture of blood-dispersible gas and sclerosant liquid, whereby upon release of the mixture the components of the mixture interact to form a microfoam, characterized in that the physiologically acceptable blood-dispersible gas is stored in the presence of one or more detectable gases selected from helium, neon, xenon, argon, sulfur hexafluoride and nitrous oxide, where the total volume of detectable gases comprises from 0.01% to 40% of the total volume of gas. The pressurized gas mixture may be stored long term in the same container as the aqueous sclerosant liquid, if long term stability tests show no degradative reaction between the gas mixture and the aqueous sclerosant liquid.

Alternatively the oxygen component of the final gas mix is stored in a separate container from the aqueous sclerosant liquid and introduced immediately prior to use. The oxygen component of the gas may thereby be stored in a container provided with engaging means for the container holding the aqueous sclerosant liquid. Such an engaging means is disclosed in WO 02/41872-A1.

In a third aspect of the present invention there is provided a device for producing a microfoam suitable for use in scleropathy of blood vessels, comprising a housing in which is situated a pressurisable chamber containing a solution of the sclerosing agent in a physiologically acceptable solvent; a pathway with one or more outlet orifices by which the solution may pass from the pressurisable chamber to the exterior of the device through said one or more outlet orifices and a mechanism by which the pathway from the chamber to the exterior can be opened or closed such that, when the container is pressurized and the pathway is open, fluid will be forced along the pathway and through the one or more outlet orifices;
said housing incorporating an inlet for the admission of a pressurized source of physiologically acceptable gas that is dispersible in blood; the gas being in contact with the solution on activation of the mechanism such as to produce a gas-solution mixture;
said pathway to the exterior of the housing including one or more foaming elements;
characterized in that the housing is charged with blood-dispersible gas stored in the presence of one or more detectable gases selected from helium, neon, xenon, argon, sulfur hexafluoride and nitrous oxide, where the total volume of detectable gases comprises from 0.01% to 40% of the total volume of gas.

In a fourth aspect of the present invention there is provided a method of treating a patient in need of sclerotherapy of a blood vessel comprising administering a microfoam as described above. There is further provided the use of such a microfoam in the manufacture of a medicament for sclerotherapy.

In all the above aspects of the invention, the detectable gas preferably comprises helium. The suitability of using helium in foam sclerotherapy techniques has already been determined by J. García Mingo. See his contribution to “Foam Sclerotherapy: State of the Art” (Editions Phébologiques Francaises), edited by Jean-Paul Henriet, pages 45-50.

The sclerosant liquid utilized in the invention may be any of those discussed in WO 00/72821-A1 and WO 02/41872-A1. Preferably the sclerosant liquid is a solution of polidocanol or sodium tetradecyl sulfate in an aqueous carrier, e.g. water, particularly in a saline. More preferably the solution is from 0.25 to 5% vol/vol polidocanol, preferably in sterile water or a physiologically acceptable saline, e.g. in 0.5 to 2% vol/vol saline. More preferably still, the concentration of polidocanol is from 0.5 to 5% vol/vol in the liquid, preferably 0.5 to 3% vol/vol polidocanol and most preferably being 1% vol/vol in the liquid. Concentration of sclerosant in the solution will be advantageously increased for certain abnormalities such as Klippel-Trenaunay syndrome.

The sclerosant may also contain additional components, such as stabilizing agents, e.g. foam stabilizing agents, e.g. such as glycerol. Further components may include alcohols such as ethanol. Even though this can reduce foam stability, inclusion of a few percent of ethanol is thought to aid in solubilizing low-molecular-weight oligomers of polidocanol and also prevent degradation of the polidocanol.

The water or saline also may contain 2-5% vol/vol physiologically acceptable alcohol, e.g. ethanol. The polidocanol solution is preferably phosphate buffered.

Addition of glycerol to the aforesaid sclerosant imparts a longer half-life to the resultant foam.

For the purpose of this application terms have the following definitions. Physiologically acceptable blood dispersible gas is a gas that is capable of being substantially completely dissolved in or absorbed by blood. A sclerosant liquid is a liquid that is capable of sclerosing blood vessels when injected into the vessel lumen. Scleropathy or sclerotherapy relates to the treatment of blood vessels by injection of a sclerosing agent to eliminate them. An aerosol is a dispersion of liquid in gas. Half-life of a microfoam is the time taken for half the liquid in the microfoam to revert to unfoamed liquid phase, under the influence of gravity, and at a defined temperature.

The mixture of blood-dispersible gas and sclerosant liquid is preferably pressurized to a pre-determined level. Preferred pressures are in the range 800 mbar to 4.5 bar gauge (1.8 bar to 5.5 bar absolute). Pressures in the range of 1 bar to 2.5 bar gauge have been found to be particularly effective over this range of pressures, there is very little change in either the density or the half-life of the resulting foam as the canister empties.

Preferably the microfoam is such that less than 20% of the bubbles are less than 30 μm diameter, greater than 75% are between 30 and 280 μm diameter, less than 5% are between 281 and 500 μm diameter, and there are substantially no bubbles greater than 500 μm diameter.

Preferably the gas/liquid ratio in the mix is controlled such that the density of the microfoam is 0.07 g/ml to 0.19 g/ml, more preferably 0.10 g/ml to 0.15 g/ml.

Preferably the microfoam has a half-life of at least 2 minutes, more preferably at least 2.5 minutes. The half-life may be as high as 1 or 2 hours or more, but is preferably less than 60 minutes, more preferably less than 15 minutes and most preferably less than 10 minutes.

The present invention will now be described further by way of illustration only by reference to the following Figures and Examples. Further embodiments falling within the scope of the invention will occur to those skilled in the art in the light of these.

### FIGURES

FIG. 1 shows a cross-sectional view of a pre-pressurized container for the generation of therapeutic microfoam according to the invention, as disclosed in WO 00/72821-A1 and further described in Example 1 below.

FIG. 2 shows a shows a cross-sectional view of a device comprising a container provided with engaging means and a mesh stack shuttle according to the invention, as disclosed in WO 02/41872-A1 and further described in Example 2 below.

FIG. 3 shows an apparatus for use in the helium detection technique as further described in Example 3 below.

### EXAMPLES

### Example 1

### Pre-Pressurized Container

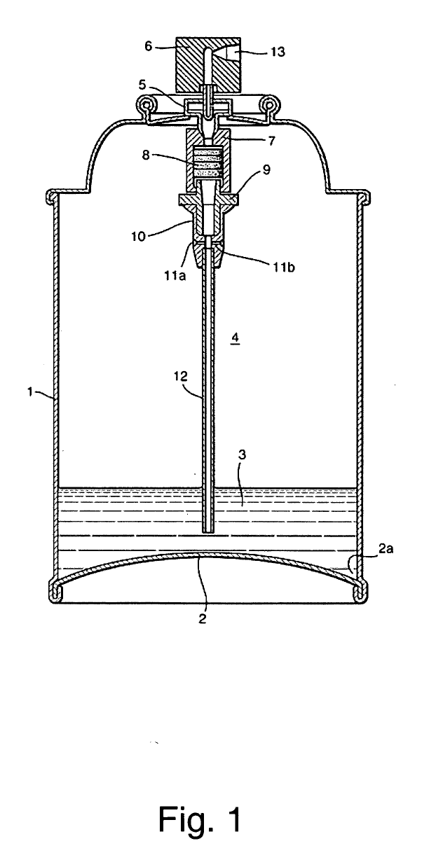

A typical apparatus for the generation of therapeutic microfoam according to the invention, as disclosed in WO 00/72821-A1, is shown in FIG. 1.

The canister has an aluminum wall (1), the inside surface of which is coated with an epoxy resin. The bottom of the canister (2) is domed inward. The canister inner chamber (4) is pre-purged with 100% oxygen for 1 minute, containing 15 ml of a 1% vol/vol polidocanol/20 mmol phosphate buffered saline solution/4% ethanol, of composition as given in Table 1 below, then filled with an oxygen-helium mixture at 2.7 bar gauge (1.7 bar over atmospheric). This is provided by introducing a charge of helium and then overpressuring the polidocanol part filled can with 1.7 bar oxygen.

A typical gas mixtures is 3% He, 25 and 35% CO₂, with the balance O₂ as a final gas mixture at approx 3.5 bar absolute.

A standard 1 inch diameter EcoSol™ aerosol valve (5) (Precision Valve, Peterborough, UK) is crimped into the top of the canister after sterile part filling with the solution and may be activated by depressing an actuator cap (6) to release content via an outlet nozzle (13) sized to engage a Luer fitting of a syringe or multi-way connector (not shown). A further connector (7) locates on the bottom of the standard valve and mounts four Nylon 66 meshes held in high density polyethylene (HDPE) rings (8), all within an open-ended polypropylene casing. These meshes have diameter of 6 mm and have a 14% open area made up of 20 μm pores, with the meshes spaced 3.5 mm apart.

A further connector (9) locates on the bottom of the connector holding the meshes and receives a housing (10) which mounts the dip tube (12) and includes gas receiving holes (11a, 11b) which admit gas from chamber (4) into the flow of liquid which rises up the dip-tube on operation of the actuator (6). These are conveniently defined by an Ecosol™ device provided by Precision Valve, Peterborough, UK, provided with an insert. Holes (11a, 11b) have cross-sectional area such that the sum total ratio of this to the cross-sectional area of the liquid control orifice at the base of the valve housing (at the top of the dip-tube) is controlled to provide the required gas/liquid ratio.

### Example 2

### Container with Engaging Means and Mesh Stack Shuttle

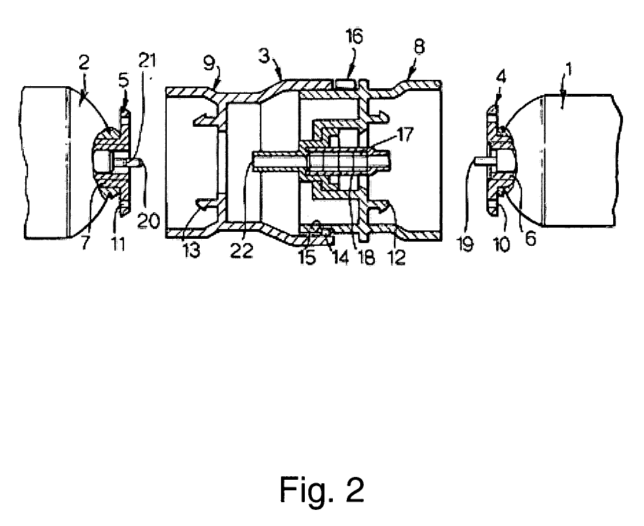

A device comprising a container provided with engaging means and a mesh stack shuttle according to the invention, as disclosed in WO 02/41872-A1, is shown in FIG. 2. The device comprises a low pressure container (1) for an aqueous sclerosant liquid and an unreactive gas atmosphere, a container (2) for a physiologically acceptable blood-dispersible gas and an engaging means comprising a connector (3).

The container (2) for a physiologically acceptable blood-dispersible gas is charged at 5.8 bar absolute pressure with an oxygen-helium mixture containing 3% helium, whereas the container (1) is charged with a carbon dioxide-helium mixture containing 3% helium. Container (2) is used to pressurize container (1) at the point of use to approx 3.5 bar absolute and is then discarded, just before the microfoam is required. The two containers will thus be referred to hereinafter as the PD [polidocanol] can (1) and the O₂ can (2).

Each of the cans (1, 2) is provided with a snap-fit mounting (4, 5). These may be made as identical moldings. The snap-fit parts (4, 5) engage the crimped-on mounting cup (6, 7) of each can (1, 2) with high frictional force. The connector is made in two halves (8, 9), and the high frictional force allows the user to grip the two connected cans (1, 2) and rotate the connector halves (8, 9) relative to each other without slippage between connector (3) and cans. Each of these can mountings (6, 7) has snap-fit holes (10, 11) for engaging mating prongs (12, 13) which are on the appropriate surfaces of the two halves (8, 9) of the connector.

The connector (3) is an assembly comprising a number of injection moldings. The two halves (8, 9) of the connector are in the form of cam track sleeves which fit together as two concentric tubes. These tubes are linked by proud pins (14) on one half that engage sunken cam tracks (15) on the other half. The cam tracks have three detented stop positions. The first of these detents is the stop position for storage. An extra security on this detent is given by placing a removable collar (16) in a gap between the end of one sleeve and the other. Until this collar (16) is removed it is not possible to rotate the sleeves past the first detent position. This ensures against accidental actuation of the connector.

The cam track sleeves (8, 9) are injection molded from ABS as separate items, and are later assembled so that they engage one another on the first stop of the detented cam track. The assembled sleeves are snap-fitted as a unit onto the O₂ can (2) mounting plate (5) via four locating prongs. The security collar is added at this point to make an O₂ can subassembly.

The connector (3) includes in its interior a series of foaming elements comprising a mesh stack shuttle (17) on the connector half (8) adjacent to the PD can (1). The mesh stack shuttle (17) is comprised of four injection molded disk filters with mesh hole size of 20 μm and an open area of approx. 14%, and two end fittings, suitable for leak-free connection to the two canisters. These elements are pre-assembled and used as an insert in a further injection molding operation that encases them in an overmolding (18) that provides a gas-tight seal around the meshes, and defines the outer surfaces of the mesh stack shuttle. The end fittings of the stack (17) are designed to give gas-tight face and/or rim seals against the stem valves (19, 20) of the two cans (1, 2) to ensure sterility of gas transfer between the two cans.

The mesh stack shuttle (17) is assembled onto the PD can valve (19) by push-fitting the components together in a aseptic environment.

The PD can (1) and attached shuttle (17) are offered up to the connector (3) and the attached O₂ can (2), and a sliding fit made to allow snap-fitting of the four locating prongs (12) on the PD can side of the connector (3) into the mating holes (10) in the mounting plate (4) on the PD can (1). This completes the assembly of the system. In this state, there is around 2 mm of clearance between the stem valve (20) of the O₂ can (2) and the point at which it will form a seal against a female Luer outlet from the stack.

When the security collar (16) is removed, it is possible to grasp the two cans (1, 2) and rotate one half of the connector (3) against the other half to engage and open the O₂ can valve (20).

As the rotation of the connector (3) continues to its second detent position, the PD can valve (19) opens fully. The gas flow from the O₂ can (2) is restricted by a small outlet hole (21) in the stem valve (20). It takes about 45 seconds at the second detent position for the gas pressure to (almost) equilibrate between the two cans to a level of 3.45 bar ±0.15 bar.

After the 45 second wait at the second detent position, the connector (3) is rotated further to the third detent position by the user. At this position, the two cans (1, 2) can be separated, leaving the PD can (1) with half (8) of the connector and the shuttle assembly (17) captive between the connector and the PD can. The O₂ can (2) is discarded at this point.

A standard 1 inch diameter aerosol valve (19) (Precision Valve, Peterborough, UK) is crimped into the top of the PD can (1) before or after sterile filling with the solution and may be activated by depressing the mesh stack shuttle (17), which functions as an aerosol valve actuator mechanism, to release the contents via an outlet nozzle (22) sized to engage a Luer fitting of a syringe or multi-way connector (not shown).

### Example 3

### Helium Detection Technique

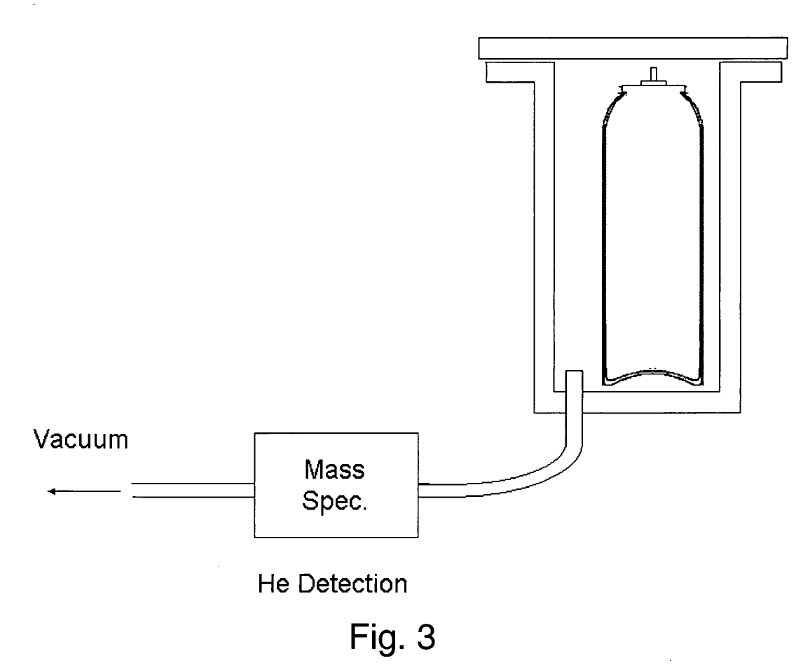

A leak detector incorporating an apparatus for the generation of therapeutic microfoam according to the invention is shown in FIG. 3. The device uses a commercially available leak detector, the Veeco™ MS-40 portable automatic leak detector, provided by the Vacuum Instrument Corporation, Ronkonkoma, N.Y.

The leak detector uses a large capacity internal mechanical pump and a mass spectrometer comprising a 180-degree deflection dual magnetic sector mass spectrometer tube with built-in high vacuum ion gauge. The mass spectrometer is sensitive to Helium Mass 3 or Mass 4 and is operator selectable.

An apparatus for the generation of therapeutic microfoam according to the invention, such as described in Examples 1 or 2, is placed is a sealed chamber. The space between the generator and the sealed chamber is then evacuated using the internal mechanical pump, and helium levels, emanating from the generator into the sealed, evacuated space around it, are detected using the mass spectrometer.

TABLE 1
Composition of 1% Polidocanol solution

| | Quantities | |
|---|---|---|
| Material | % ^{w}/_{w} | per 1000 g |
| Polidocanol | 1.000 | 10.00 g |
| Ethanol 96% EP | 4.200 | 42.00 g |
| Disodium Hydrogen Phosphate | 0.240 | 2.40 g |
| Dihydrate. EP | | |
| Potassium Di-hydrogen | 0.085 | 0.85 g |
| Phosphate. EP | | |
| 0.1 M Sodium Hydroxide | q.s. | q.s. |
| Solution [used for | | |
| adjustment of pH: 7.2-7.5] | | |
| 0.1 M Hydrochloric Acid | q.s. | q.s. |
| Water for injection. EP [used | approx. 94.475 q.s. | approx. 944.75 g |
| to adjust to final | to 100.00% | q.s. to 1000.00 g |
| weight] | | |
| TOTAL: | 100.00% | 1000.00 g |

1. A sclerosing foam comprising a physiologically acceptable gas that is readily dispersible in blood together with an aqueous sclerosant liquid, in which the foam is a microfoam further including one or more detectable gases selected from helium, neon, xenon, argon, sulfur hexafluoride and nitrous oxide, where the total volume of detectable gases comprises from 0.01% to 40% of the total volume of gas.

2. A sclerosing foam as claimed in claim 1, in which the microfoam includes detectable gases in an amount from 0.1% to 40% of the total volume of gas.

3. A sclerosing foam as claimed in claim 2, in which the microfoam includes detectable gases in an amount from 0.5% to 20% of the total volume of gas.

4. A sclerosing foam as claimed in claim 3, in which the microfoam includes detectable gases in an amount from 1% to 10% of the total volume of gas.

5. A sclerosing foam as claimed in claim 4, in which the microfoam includes detectable gases in an amount from 1% to 5% of the total volume of gas.

6. A sclerosing foam as claimed in claim 1, in which the foam is made from a gas mixture including less than 10% vol/vol nitrogen.

7. A sclerosing foam as claimed in claim 1, in which the foam is made from a gas mixture comprising at least 50% of the physiologically acceptable gases oxygen and/or carbon dioxide.

8. A sclerosing foam as claimed in claim 7, in which the gas mixture comprising at least 75% oxygen and/or carbon dioxide.

9. A sclerosing foam as claimed in claim 8, in which the gas mixture comprises at least 99% oxygen or carbon dioxide.

10. A sclerosing foam as claimed in claim 1, in which the sclerosant liquid is a solution of polidocanol or sodium tetradecyl sulfate in an aqueous carrier.

11. A sclerosing foam as claimed in claim 10, in which the sclerosant liquid is a solution of 0.25 to 5% vol/vol polidocanol.

12. A sclerosing foam as claimed in claim 1, in which the microfoam is such that less than 20% of the bubbles comprising the microfoam are less than 30 μm diameter, greater than 75% are between 30 and 280 μm diameter, less than 5% are between 281 and 500 μm diameter, and there are substantially no bubbles greater than 500 μm diameter.

13. A sclerosing foam as claimed in claim 1, in which the gas/liquid ratio in the mix is controlled such that the density of the microfoam is 0.07 g/ml to 0.19 g/ml.

14. A sclerosing foam as claimed in claim 1, in which the microfoam has a half-life of at least 2 minutes.

15. A sclerosing foam as claimed in claim 1, in which the detectable gas comprises helium.

16. A method for producing a microfoam suitable for use in scleropathy of blood vessels, comprising introducing a physiologically acceptable blood-dispersible gas into a container holding an aqueous sclerosant liquid and releasing the mixture of blood-dispersible gas and sclerosant liquid, whereby upon release of the mixture the components of the mixture interact to form a microfoam, in which the physiologically acceptable blood-dispersible gas is stored in the presence of one or more detectable gases selected from helium, neon, xenon, argon, sulfur hexafluoride and nitrous oxide, where the total volume of detectable gases comprises from 0.01% to 40% of the total volume of gas.

17. A method as claimed in claim 16, in which the oxygen component of the final gas mix is stored in a separate container from the aqueous sclerosant liquid and introduced immediately prior to use.

18. A sclerosing foam as claimed in claim 16, in which the detectable gas comprises helium.

19. A device for producing a microfoam suitable for use in scleropathy of blood vessels, comprising a housing in which is situated a pressurisable chamber containing a solution of the sclerosing agent in a physiologically acceptable solvent; a pathway with one or more outlet orifices by which the solution may pass from the pressurisable chamber to the exterior of the device through said one or more outlet orifices and a mechanism by which the pathway from the chamber to the exterior can be opened or closed such that, when the container is pressurized and the pathway is open, fluid will be forced along the pathway and through the one or more outlet orifices;
said housing incorporating an inlet for the admission of a pressurized source of physiologically acceptable gas that is dispersible in blood; the gas being in contact with the solution on activation of the mechanism such as to produce a gas-solution mixture;
said pathway to the exterior of the housing including one or more foaming elements;
in which the housing is charged with blood-dispersible gas stored in the presence of one or more detectable gases selected from helium, neon, xenon, argon, sulfur hexafluoride and nitrous oxide, where the total volume of detectable gases comprises from 0.01% to 40% of the total volume of gas.

20. A sclerosing foam as claimed in claim 16, in which the detectable gas comprises helium.

21. A method of treating a patient in need of sclerotherapy of a blood vessel comprising administering a sclerosing foam as claimed in claim 1.

22. A method of treating a patient in need of sclerotherapy of a blood vessel comprising administering a sclerosing foam as claimed in claim 1, in which the detectable gas comprises helium.
